(19) United States
(12) Patent Application Publication
(10) Pub. No.: US 2012/0003176 A1
(43) Pub. Date: Jan. 5, 2012

(54) HYDROGEL-FORMING COMPOSITION COMPRISING NATURAL AND SYNTHETIC SEGMENTS

(75) Inventors: Xiao Huang, Freehold, NJ (US); Jian Q. Yao, Shanghai (CN); Jizong Gao, Cedar Park, TX (US); Victor Zaporojan, Richardson, TX (US); Joe Zhe Guo, Freehold, NJ (US)

(73) Assignee: Zimmer Orthobiologics, Inc., Austin, TX (US)

(21) Appl. No.: 13/174,121

(22) Filed: Jun. 30, 2011

Related U.S. Application Data
(60) Provisional application No. 61/361,182, filed on Jul. 2, 2010.

(51) Int. Cl. A61K 31/785; A61K 35/12; A61L 31/14; A61K 47/36; A61L 31/04

(52) U.S. Cl. 424/78.27; 514/772.2; 523/113; 424/93.7; 514/769

(57) ABSTRACT

A hydrogel composition is formed from a natural polymer derivative having a plurality of cross-linkable units depending therefrom, and a synthetic polymer derivative having a plurality of cross-linkable units depending therefrom, said natural and synthetic polymer derivatives having hydrolysable units disposed between the polymer backbone of said derivative and at least some of said cross-linkable units. The use of a combination of natural and synthetic polymers provides for biodegradability of the hydrogel, along with mechanical properties such as strength and elasticity.

### CROSS-REFERENCE TO RELATED APPLICATIONS

This application claims the benefit of U.S. Provisional Application No. 61/361,182, filed on Jul. 2, 2010, under 35 U.S.C. §119(e), which is incorporated herein by reference in its entirety.

### BACKGROUND OF THE INVENTION

The term “hydrogel” refers to a broad class of polymeric materials which are swollen extensively in water, but which do not dissolve in water. Generally, hydrogels are formed by polymerizing a hydrophilic monomer in an aqueous solution under conditions where the polymer becomes cross-linked so that a three-dimensional polymer network is formed which is sufficient to gel the solution.

Hydrogels have many desirable properties for biomedical applications. For example, they can be made nontoxic and compatible with tissue. In addition, they are usually highly permeable to water, ions and small molecules. It is known to make hydrogels of synthetic materials such as polyvinyl alcohols.

U.S. Pat. No. 6,497,903 to Hennink, incorporated herein by reference in its entirety, purports to disclose a biodegradable hydrogel having a network of polymer chains, wherein the polymer chains are interconnected to one another through spacers, the spacers comprising a cross-linking unit and one or more hydrolysable bonds, the cross-linking unit being derived from a hydroxyl-alkyl methacrylate unit, the hydrolysable bonds being selected from carbonate, carboxylic acid ester, urethane, anhydride, (hemi)acetal, and amide bonds, and the polymer chains being based on dextran or derivatized dextran. U.S. Pat. No. 6,497,903 also mentions hydrogels having cross-linked dextrans obtained by coupling glycidyl methacrylate (GMA) to dextran, followed by radical polymerization of an aqueous solution of GMA-derivatized dextran (dex-GMA), referring to Van Dijk-Wolthuis et al. in Macromolecules 28, (1995), 6317-6322 and to De Smedt et al. in Macromolecules-28, (1995) 5082-5088.

In the healing of localized injury or disease, localized delivery of therapeutic agents can be advantageous compared to systemic delivery of therapeutic agents, because systemic delivery can require much higher doses of the therapeutic agent and can result in undesirable side effects. Thus medical research has been directed to means and methods for localized delivery of therapeutic agents. Such means and methods include, for example, bolus injection, minipump delivery, and injectable gels.

### SUMMARY OF THE INVENTION

This invention relates to hydrogel-forming compositions usable as biomedical implants or media for promoting healing and/or tissue regeneration. This invention further relates to hydrogel-forming compositions usable as biomedical implants or media that can contain therapeutic agents, to provide therapeutic agents to areas of the body in need of healing and/or tissue regeneration.

Thus, the invention provides a biocompatible composition and method of delivering therapeutic agents to different parts of the body, e.g., delivering therapeutic agents that biodegrade in situ and which may be formulated to biodegrade at a predetermined rate. For example, joint structures can undergo injury or damage such as osteoarthritis in the cartilage and meniscal tissue degeneration and the hydrogel-forming compositions or hydrogel compositions may be administered to joint structures to provide a biocompatible three-dimensional structure that promotes healing and/or tissue regeneration. The hydrogels of the invention provide for local delivery of therapeutic agents such as pharmaceutical agents, growth factors, or cells directly to a site requiring treatment, e.g., to create an optimal regeneration environment.

In accordance with one aspect of the invention, a hydrogel-forming composition includes a natural polymer derivative having a plurality of cross-linkable units depending therefrom, and a synthetic polymer derivative having a plurality of cross-linkable units depending therefrom, said natural and synthetic polymer derivatives having hydrolysable units disposed between the polymer backbone of said derivative and at least some of said cross-linkable units.

The composition can form a hydrogel upon initiation of cross-linking between the cross-linkable units on the natural and synthetic polymer molecules, whereupon the cross-linked natural and synthetic polymers form a three-dimensional structure that swells but does not dissolve in water.

In accordance with another aspect of the invention, a hydrogel composition includes the cross-linked product of a natural polymer derivative having a plurality of cross-linkable units depending therefrom, and a synthetic polymer derivative having a plurality of cross-linkable units depending therefrom, said natural and synthetic polymer derivatives having hydrolysable units disposed between the polymer backbone of said derivative and at least some of said cross-linkable units.

The choices of natural and synthetic polymers, cross-linkable units, and hydrolysable units will be factors in determining the properties of the hydrogel-forming composition, the properties of the hydrogel formed from the composition, and the rate of degradation of the hydrogel formed from the composition.

In accordance with another embodiment of the invention, a method of making a hydrogel composition includes providing a natural polymer derivative having a plurality of cross-linkable units depending therefrom, providing a synthetic polymer derivative having a plurality of cross-linkable units depending therefrom, said natural and synthetic polymer derivatives having hydrolysable units disposed between the polymer backbone of said derivatives and at least some of said cross-linkable units, and cross-linking said cross-linkable units depending from said natural polymer derivative with said cross-linkable units depending from said synthetic polymer derivative.

In yet another embodiment of the invention, a method of making a hydrogel composition includes providing a natural polymer derivative having a plurality of cross-linkable units depending therefrom, providing a synthetic polymer derivative having a plurality of cross-linkable units depending therefrom, said natural and synthetic polymer derivatives having hydrolysable units disposed between the polymer backbone of said derivatives and at least some of said cross-linkable units, cross-linking said cross-linkable units depending from said protein polymer derivative with said cross-linkable units depending from said non-protein polymer derivative, and providing an aqueous medium, whereby the cross-linked product forms a hydrogel with an aqueous medium.

In one embodiment, one or more therapeutic agents are provided prior to the cross-linking step. In one embodiment, one or more therapeutic agents are provided after the cross-linking step. In one embodiment, an aqueous medium, e.g., phosphate buffered saline, is present during cross-linking. In one embodiment, one of the therapeutic agents is a pharmaceutically active compound. In one embodiment, one of the therapeutic agents is a population of mammalian cells.

In one embodiment, said cross-linkable units are selected from vinyl units, acrylate units, or mixtures thereof. In one embodiment, said natural polymer derivative is derived from a natural polymer selected from one or more of dextrans, polysaccharides, proteins such as gelatins and collagens, hyaluronic acid, and polyglycans. In one embodiment, said synthetic polymer derivative is derived from a synthetic polymer selected from one or more of methyl cellulose, N-Isopropylacrylamide (NiPAAM); poly(vinyl alcohol); poly(NiPAAM)/poly(ethylene glycol); poly(ethylene oxide-propylene oxide-ethylene oxide) (PEO-PPO-PEO); and Poly(ethylene glycol-lactic acid-ethylene glycol) (PEG-PLLA-PEG). In one embodiment, said hydrolysable units are selected from one or more of lactates, e.g., oligolactate, polyglycolic acid, polylactic acid, carbonate esters, carboxylic esters, urethanes, anhydrides, (hemi)acetals, amides, peptide bonds, and dimers and oligomers thereof.

The composition can be administered in the liquid state, such as by injection, to a subject in need of therapy. Either before or after administration, cross-linking can be induced (initiated) in the cross-linkable units, whereby the composition can form a hydrogel. Such cross-linking can be induced by means such as photo-initiation; other means for inducing cross-linking can be selected based upon the type of cross-linkable units employed. Thus, in one embodiment of the invention, a method of administering a hydrogel composition to provide therapy to a site in need thereof includes providing a hydrogel-forming composition as described, administering said hydrogel-forming composition to the site, and initiating cross-linking of said hydrogel-forming composition to form a hydrogel.

In one embodiment, the invention provides an injectable hydrogel-forming composition that is suitable for injections either in the intraarticular region or in sites other than the intraarticular region. In one embodiment, when the hydrogel-forming composition comprises a therapeutic agent, e.g., when the therapeutic agent to be delivered by the injectable hydrogel is cells, the hydrogel-forming composition may improve cell adhesion, improve cell proliferation, improve cell differentiation, and/or improve new tissue matrix formation in the hydrogel environment. In one embodiment, a hydrogel-forming composition for the delivery of therapeutic agents, including cells, comprises a gel that is biocompatible and biomimetic, thereby providing biological clues to the therapeutic cells being delivered and to the surrounding tissue.

In one embodiment, the invention provides a biomedically compatible hydrogel material that can serve as a scaffolding or void filler to promote the regeneration of tissue at an affected site, either with or without one or more added therapeutic agents.

In one embodiment, the invention provides an injectable hydrogel-forming composition that can form a hydrogel in situ upon injection.

In one embodiment, the invention provides an injectable hydrogel-forming composition that can be injected as a hydrogel.

In one embodiment, the invention provides an injectable hydrogel-forming composition that either can form a hydrogel in situ upon injection, or can be injected as a hydrogel, which composition is capable of delivering therapeutic agents to a site in need thereof.

In one embodiment, the invention provides an injectable hydrogel-forming composition that can form a hydrogel in situ upon injection, or can be injected as a hydrogel, and which hydrogel is biodegradable by hydrolysis and/or enzyme activity.

In one embodiment, the invention provides an injectable hydrogel-forming composition that forms a hydrogel in situ upon injection or can be injected as a hydrogel and which comprises both a protein polymer and a non-protein polymer.

In one embodiment, the invention provides a method of preparing a hydrogel-forming composition.

In one embodiment, the invention provides a method of using a hydrogel-forming composition to provide one or more therapeutic agents to a site in need of such agents.

In another embodiment of the invention, a method is provided that includes administering a hydrogel composition to provide therapy to a site in need thereof. The method includes providing a hydrogel-forming composition as described, initiating cross-linking of said hydrogel-forming composition to form a hydrogel composition, and administering said hydrogel composition to said site.

The hydrogel remains at the site of application for a period of time in which it can provide a therapeutic effect. For example, the hydrogel can provide a scaffold structure to support the generation of tissue at the site of application. Further, in any of the foregoing embodiments, the hydrogel composition or hydrogel-forming composition optionally can comprise an amount of one or more therapeutic agents. The hydrogel remains at the site of application for a time sufficient to allow at least some of the therapeutic agent carried by the gel to be in contact with the site in need of therapy.

Over time, the hydrolysable units disposed between the natural and synthetic polymer backbones and their respective cross-linkable units are hydrolyzed under the ambient conditions in the body. This hydrolysis of the hydrolysable units will cause the hydrogel to biodegrade into the individual polymer chains, and chains of cross-linkable units. These individual chains, no longer linked together in a three-dimensional structure, will eventually be eliminated from the body. Thus, the hydrogel can remain in the desired location to provide some therapeutic effect, and/or until at least a portion of the optional therapeutic agent therein has been delivered to the affected site in the patient, and then biodegrade. The rate of biodegradation can be controlled by the choices of natural polymer, synthetic polymer, cross-linkable units, and hydrolysable units.

### DESCRIPTION OF THE FIGURES

FIG. 1 is a schematic representation of the cross-linking between natural and synthetic polymer derivatives that occurs to form the hydrogels of the present invention, and the subsequent hydrolysis of the hydrogels upon biodegradation.

FIG. 2 shows photographs of chondrocytes encapsulated in a hydrogel of the present invention after culturing for one week and three weeks.

FIG. 3 shows gene expression of chondrocytes encapsulated in a hydrogel of the present invention after culturing for one week, two weeks, and three weeks.

### DETAILED DESCRIPTION OF THE INVENTION

A hydrogel-forming composition of one embodiment of the present invention has polymer derivatives formed from a natural polymer derivative and a synthetic polymer derivative, the polymer derivatives having cross-linkable units depending therefrom, with hydrolysable units disposed between at least some of the cross-linkable units and the polymer backbones. The hydrogel-forming composition optionally comprises one or more therapeutic agents. The composition may be in a liquid state when first formed. The composition forms a hydrogel when the cross-linking of the cross-linkable units, where the natural and synthetic polymer chains form a cross-linked structure that can absorb and retain a significant proportion of water.

The hydrogels formed by the compositions and methods of the present invention advantageously undergo degradation in situ by the action of hydrolysis of the hydrolysable units of the hydrogel. It is thus possible for the hydrogel formulation to manipulate the degradation rate of the hydrogel to suit a particular application by varying the choices of the hydrogel components and the chemistry of their assembly into a hydrogel. The choices of components also can be used to adjust the mechanical properties of the hydrogel. The hydrogel may function as a scaffold for the generation of tissue at an affected site. Hydrogels of the invention may also contain one or more therapeutic agents such as cells or pharmaceuticals, to serve as delivery vehicles of such therapeutic agents to a site in need thereof.

Thus, in one embodiment the hydrogel formed from the hydrogel-forming composition comprises the cross-linked product of a natural polymer derivative having a plurality of cross-linkable units depending therefrom, and a synthetic polymer derivative having a plurality of cross-linkable units depending therefrom, said natural and synthetic polymer derivatives having hydrolysable units disposed between the polymer backbone of said derivative and at least some of said cross-linkable units.

Types of polymers that can be used in hydrogel-forming compositions of the present invention include synthetic polymers, natural protein polymers, and natural non-protein polymers. Compositions within the scope of the present invention include, without limitation, mixtures of synthetic polymer derivatives with protein polymer derivatives, and mixtures of synthetic polymer derivatives with non-protein natural polymer derivatives. The word “derivative” as used herein refers to the polymer chains having cross-linkable units attached thereto, or cross-linkable and hydrolysable units attached thereto. For purposes of this application, DS indicates the degree of substitution. For example, the DS may be the amount of methacrylate groups per 100 dextran glycopyranose residues. DPav indicates average degree of polymerization, e.g., the average length of a spacer, for instance, a lactate spacer.

The synthetic polymer component of the hydrogel structure can be selected to impart desirable mechanical properties to the hydrogel structure that may not be present when purely natural biodegradable polymers are used. For example, properties such as strength and elasticity can be improved by the presence of synthetic polymers. Synthetic polymers suitable for use in the present invention can be either thermo-sensitive or non-thermosensitive, depending on the application in which they are used. Synthetic thermosensitive polymers suitable for use in the present invention include, but are not limited to, for example, methylcellulose, N-Isopropylacrylamide (poly-NiPAAM); and poly(ethylene oxide-propylene oxide-ethylene oxide) (PEO-PPO-PEO). Synthetic non-thermosensitive polymers suitable for use in the present invention include, for example, polymers such as poly(vinyl alcohol); poly(ethylene glycol-lactic acid-ethylene glycol) (PEG-PLLA-PEG); polyethylene glycols; polyesters; polyanhydrides; polyurethanes; and blends, co-polymers, and block polymers of any two or more of the foregoing.

Natural non-protein polymers suitable for use in the present invention include, but are not limited to, for example, polysaccharides such as dextran, hyaluronic acid, polyglycans, cellulose, chitosan, and alginate. Protein polymers suitable for use as the natural polymer in the present invention may be extracellular matrix proteins, including structural proteins such as collagen Type I, collagen Type II, and elastin; specialized proteins such as fibrillin, fibronectin, and laminin; proteoglycans, e.g., combinations of carbohydrates and proteins; and gelatins, including Type A gelatins, type B gelatins, and mixtures thereof, and native gelatins from sources such as bovine skin, porcine skin, and fish skin. Hydrogel compositions of the present invention also can include combinations of any two or more of the foregoing types of protein or non-protein polymers.

Those skilled in the art of hydrogels will recognize other natural polymers suitable for use in the present invention.

Both the protein polymers and the non-protein polymers of the invention may be provided with cross-linkable units. Suitable cross-linkable units can include for example, vinyl units such as in vinyl ethers or vinyl esters, acrylic units such as in acrylates or methacrylates, or mixtures thereof. In one embodiment the cross-linkable unit comprises hydroxyethylmethacrylate (“HEMA”). Succinates also can be used as cross-linkable units, such as N-hydroxysuccinimides and acrylate-PEG-N-hydroxysuccinimide (acrylate-PEG-NHS). Such cross-linkable moieties can be added to the polymers by methods known to those skilled in the art.

Suitable hydrolysable units can include one or more of ester units such as lactates, in particular oligolactates; polyglycolic acid, polylactic acid, carbonate esters, carboxylic esters, urethanes, anhydrides, (hemi)acetals, amides and peptide bonds also can be used, as well as dimers and oligomers thereof.

The present invention further includes a method of making a hydrogel composition. Methods of making the polymer derivatives for use in the hydrogels are generally similar to those disclosed in the previously noted U.S. Pat. No. 6,497,903, using polysaccharides in which the hydroxyl groups can be used for conjugation to yield the polymer derivatives.

In one embodiment of the method of making a hydrogel, a first solution is prepared of a natural polymer derivative, the derivative having a plurality of cross-linkable units depending from the polymer, with hydrolysable segments disposed between the polymer backbone and at least some of the cross-linkable units, and a second solution is prepared of a synthetic polymer derivative, the derivative having a plurality of cross-linkable units depending from the polymer, with hydrolysable segments disposed between the polymer and at least some of the cross-linkable units. In the case where the therapeutic agent is cells, the two solutions are combined with a quantity of cells in the presence of a cross-linking initiator to create a cell suspension. Cross-linking is then initiated to form a gel.

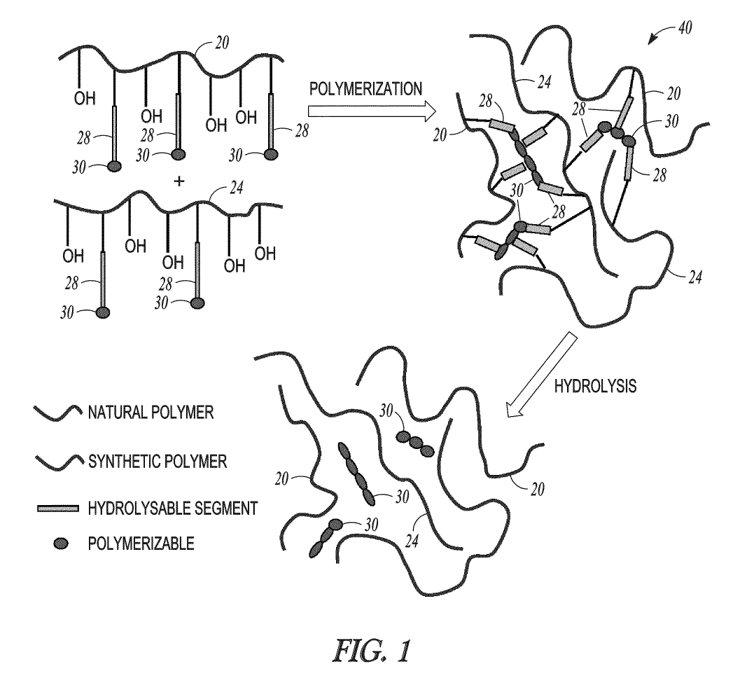

FIG. 1 is a simplified schematic representation of the cross-linking that occurs to form the hydrogels of the present invention, and their subsequent degradation by hydrolysis. As illustrated in FIG. 1, there are provided a synthetic polymer 20 and a natural polymer 24, each of which includes a plurality of hydroxyl groups arranged at various intervals along the polymer backbone. Each polymer 20, 24 further comprises a plurality of hydrolysable segments 28 attached at the sites of at least some of the hydroxyl groups. At least some of the hydrolysable segments 28 are also attached to cross-linking groups 30. The cross-linking groups attached to the natural molecules can be the same as or different from the cross-linking groups on the synthetic molecules. When cross-linking is initiated, the cross-linkable groups on the synthetic and natural polymer molecules bond with one another, thereby creating a cross-linked network 40 of synthetic polymer molecules and natural polymer molecules. In the presence of a suitable aqueous medium the network swells and forms a hydrogel. The cross-linking can occur in the presence of a therapeutic agent, or a therapeutic agent can be added to the network or the hydrogel after formation thereof.

In one exemplary method of preparing a hydrogel composition of the present invention including cells, dextran (MW of about 15K to about 20K) is modified by substituting a number of —OH groups with hydrolysable units ended with polymerizable groups, such as by the method described in Van-Dijk-Wolthius et al., Polymer, 1997, 38, 6235. The modified dextran macromer is then dissolved in Dulbecco's Phosphate Buffered Saline (DPBS) containing Irgacure 2959 (0.05 w/w % of dry polymer) at concentration of 13.3 w/v %. Also added to the DPBS is a protein polymer derivative having polymerizable groups. Similarly, polyvinyl alcohol is chemically modified and dissolved the same way as described above. The two solutions are then combined and the mixture is sterilized by filtering through 0.45 μm syringe filter. Chondrocytes are suspended in the sterilized macromer solution at 5 million cells/mL. The cell suspended macromer solution is thereafter added into the wells of a 96-well plate, 100 μm per well, and illuminated with a 4 W long-wave UV lamp for 8 minutes to form disc-shaped hydrogels. The gels are transferred to 24-well plate, washed twice with cell culture medium, and then cultured in fresh medium.

The hydrogels are delivered to a site in need of therapy, either before or after the initiation of cross-linking. The hydrogels of the present invention can be prepared and delivered to a surgical site by means of the method and apparati disclosed in co-pending published U.S. patent application Ser. Nos. 11/613,319 and 11/613,456, published as U.S. Publication No. 2008/0154233 A1 and U.S. Publication No. 2008/0154232 A1, respectively, the disclosures of each of which are incorporated herein by reference in their entireties. Other modes of delivering a hydrogel to a site in need of therapy will be recognized by those of skill in the art.

The hydrogel structure can be therapeutically beneficial by providing structural support to the surrounding tissue as a scaffold, or as a void-filler, or to supplement the viscous properties of synovial fluid within an affected joint. The hydrogel-forming composition also can serve as a delivery vehicle for one or more therapeutic agents, which can be cells, a pharmaceutical composition, or both. Depending on the particular therapeutic need, cells suitable for use as therapeutic agent can include one or more of mammalian chondrocytes, tenocytes, mesenchimal, bone marrow stem cells and other cell types. The cells may be xenogeneic, allogeneic or autogeneic to the mammal to which the hydrogel-forming composition or hydrogel is administered. When the therapeutic agent to be delivered by the injectable hydrogel is cells, and where the natural polymer is an extracellular matrix protein, then the extracellular matrix protein present in the hydrogel-forming composition may function to improve cell adhesion, improve cell proliferation, and improve cell differentiation into desired lineage in the hydrogel environment. When the natural polymer is an extracellular matrix protein it can make the hydrogel both biocompatible and biomimetic, enhancing cell activity by providing an optimal cell environment that mimics the natural extracellular matrix protein of connective tissue, thereby providing biological cues to the therapeutic cells being delivered and to the surrounding tissue; such cues can include signals for cell-matrix interactions, or growth factor receptors. It is known in the art, for example, that specific growth factors can induce specific cellular phenotype.

Incorporation of the synthetic polymer into the natural polymer hydrogel by cross-linking the two polymers through the cross-linkable units provides two benefits. First, the viscosity of the solution prior to gelation may be increased, which can improve the handling ability of the material during administration of the hydrogel do a site in need of therapy. It also was found that the degradation rate may be decreased significantly, which can prolong the therapeutic effect provided by the hydrogel with respect to scaffolding and void filling, and prolong the time period for delivery of therapeutic agents incorporated into the hydrogel.

One advantage of the hydrogel of the present invention is that the hydrogel does not need to be surgically removed when it is no longer needed at the surgical site. The hydrolysable segments will gradually be hydrolyzed over time, leading to the controlled breakdown of the hydrogel. This controlled breakdown also can result in the release of stored therapeutic agent from the hydrogel to the affected site. Referring again to FIG. 1, hydrolysis results in the cleavage of hydrolysable segments 28, leaving the individual molecules of synthetic polymer 20 and natural polymer 24, as well as polymerized strands of cross-linking agents 30. These individual molecules and strands can be carried away and excreted by the body. It will be appreciated that the rate of hydrolysis will be determined at least in part by the type of hydrolysable segment employed. Slower or faster rates of hydrolysis may be desired, depending, for example, on the nature of the therapeutic agent and its desired rate of release to the surgical site.

A representative method of practicing the present invention is set forth in the example herein, which is presented by way of illustration and not by way of limitation. Those skilled in the art will recognize that the steps described herein can be adjusted with respect to materials used, quantities, and proportions, as well as with respect to the means of initiating cross-linking of the polymers and the duration and temperature at which each step is practiced, in accordance with the particular properties desired for the completed hydrogel product.

### Example 1

In the following example, dextran is used as the natural polymer and polyvinyl alcohol is used as the synthetic polymer, but it will be understood that other natural or synthetic polymers could be used, as long as there are sufficient hydroxyl groups on the polymer backbones to serve as binding sites for the hydrolysable segments. For the dextran, succinate-lactide is used as the hydrolysable component, and acrylate units in the form of 2-hydroxyethyl methacrylate (HEMA) are used as the cross-linkable component.

Step 1. Preparation of Solution of Dex-suc-lac-HEMA

First, a complex of HEMA-lac is prepared. A solution is prepared by combining 0.728 mL (6 mmol) of 2-hydroxyethyl methacrylate (Sigma-Aldrich #477028) with 1.728 g (12 mmol) L-lactide (Sigma-Aldrich #367044) and heating in an oil bath at 110° C. with stirring under a nitrogen atmosphere until the lactide melts. Once melting occurs, 18 μL of Tin(II) 2-ethylhexanoate (0.06 mmol, 1 mol % with respect to HEMA) (Sigma-Aldrich #S3252) is added with 0.5 mL toluene, and stirring is continued at 110° C. under a nitrogen atmosphere for 45 minutes.

Next, HEMA-lac-suc is prepared by lowering the temperature of the mixture to 50° C., while maintaining stirring under a nitrogen atmosphere, then sequentially adding 0.6 g succinic anhydride (6 mmol, equimolar with HEMA)(Sigma-Aldrich #239690), 6 mL pyridine (Sigma-Aldrich # 270970), and 0.08 g 4-dimethylaminopyridine (DMAP)(Sigma-Aldrich # 107700). Stirring is continued at 50° C. under nitrogen for 2 hours, then the nitrogen is turned off and stirring is continued at 50° C. for another 15 hours.

The HEMA-lac-suc is purified by cooling the reaction mixture to room temperature, adding 4 to 5 PVA-hydrogel disks, stirring overnight, then transferring the reaction solution without the hydrogel disks. The hydrogel purification disks are prepared as follows: A 15 w/v % mixture of PVA is prepared by mixing 7.88 g poly(vinyl alcohol), MW of about 6000, 80% hydrolyzed (Polysciences #22225) with 52.5 mL deionized water. The mixture is covered and heated at about 85° C. for about one hour to form a viscous solution. The solution is cooled to room temperature, and mixed thoroughly with 1 mL 1 N HCl and 1.35 mL glutaraldehyde solution 25 wt % in H₂O (Sigma-Aldrich #G4004). The mixture is poured onto a glass plate to form a layer 3 to 5 mm thick, covered, and incubated overnight at 40° C. to form a hydrogel. The hydrogel so formed is cut into disks about 1 cm in diameter. The disks are washed in 50% ethanol for 1 day with frequent solution changes, and dried in a vacuum at 50° C. for at least 48 hours. The dried disks are stored in a dessicator until needed to purify the HEMA-lac-suc reaction product as just described.

Then, dex-suc-lac-HEMA is prepared. The purified HEMA-lac-suc is mixed under nitrogen atmosphere with a solution of 4.68 g dextran MW 15,000-20,000 (Polysciences #01341-100) in 25 mL DMSO. To this mixture is added a dispersion of 1.856 g dicyclohexylcarbodiimide (DCC, Sigma-Aldrich #D80002) in 5 mL DMSO. Stirring under nitrogen atmosphere is continued for seven hours. The mixture was vacuum filtered, and the filtrate is precipitated in 400 mL of a solvent mixture of 4:1 by volume 2-propanol and diethyl ether. The precipitate was collected by vacuum filtration and washed twice with 100 mL of the solvent mixture. The filtered product is dried at 40° C. for at least 24 hours.

The resulting derivative is characterized as DPav=6 and DS=33. The derivative is then mixed into phosphate buffered saline solution containing 0.05 w/w % Irgacure® 2959 photoinitiator available from Ciba, Inc, the polymer derivative being present in the solution in the amount of 13.3 weight percent.

Step 2. Preparation of Solution of PVA-suc-lac-HEMA

In the following step, the sources of all materials used are the same as described in step 1, except where otherwise noted.

First, a complex of HEMA-lac is prepared. A solution is prepared by combining 0.728 mL (6 mmol) of 2-hydroxyethyl methacrylate (Sigma-Aldrich #477028) with 1.728 g (12 mmol) L-lactide (Sigma-Aldrich #367044) and heated in an oil bath at 110° C. with stirring under a nitrogen atmosphere until the lactide melted. Once melting occurs, 18 μL of Tin(II) 2-ethylhexanoate (0.06 mmol, 1 mol % with respect to HEMA) (Sigma-Aldrich #S3252) is added with 0.5 mL toluene, and stirring was continued at 110° C. under a nitrogen atmosphere for about 75 minutes.

Next, HEMA-lac-suc is prepared by lowering the temperature of the mixture to 50° C., while maintaining stirring under a nitrogen atmosphere, then sequentially adding 0.6 g succinic anhydride (6 mmol, equimolar with HEMA) (Sigma-Aldrich #239690), 6 mL pyridine (Sigma-Aldrich # 270970), and 0.08 g 4-dimethylaminopyridine (DMAP) (Sigma-Aldrich #107700). Stirring is continued at 50° C. under nitrogen for 2 hours, then the nitrogen is turned off and stirring is continued at 50° C. for another 15 hours.

The HEMA-lac-suc is purified by cooling the reaction mixture to room temperature, adding 4-5 PVA-hydrogel disks, stirring overnight, then transferring the reaction solution without the hydrogel disks. The hydrogel purification disks are prepared as follows: A 15 w/v % mixture of PVA is prepared by mixing 7.88 g poly(vinyl alcohol), MW of about 6000, 80% hydrolyzed (Polysciences #22225) with 52.5 ml deionized water. The mixture is covered and heated at about 85° C. for about one hour to form a viscous solution. The solution is cooled to room temperature, and mixed thoroughly with 1 mL 1N HCl and 1.35 mL glutaraldehyde solution 25 wt % in H₂O (Sigma-Aldrich #G4004). The mixture is poured onto a glass plate to form a layer 3-5 mm thick, covered, and incubated overnight at 40° C. to form a hydrogel. The hydrogel so formed is cut into disks about 1 cm in diameter. The disks are washed in 50% ethanol for 1 day with frequent solution changes, and dried in a vacuum at 50° C. for at least 48 hours. The dried disks are stored in a dessicator until needed to purify the HEMA-lac-suc reaction product as just described.

Then, PVA-suc-lac-HEMA is prepared. The purified HEMA-lac-suc is mixed under nitrogen atmosphere with a solution prepared by dissolving 3.144 g PVA MW 6,000 (Polysciences, Inc., #22225-500) in about 30 mL DMSO with heating and stirring. To this mixture is added a dispersion of 1.856 g dicyclohexylcarbodiimide (DCC, Sigma-Aldrich #D80002) in about 5 mL DMSO. Stirring under nitrogen atmosphere at room temperature is continued for about 5 hours. The mixture is vacuum filtered, and the filtrate is precipitated in 400 mL of a solvent mixture of 4:1 by volume acetone and diethyl ether. The precipitate is collected by vacuum filtration and washed twice with 100 mL of the solvent mixture. The filtered product is dried at 40° C. for at least 24 hours.

Step 3. Preparation of Hydrogels

A first solution of 13.3 w/v % of Dex-suc-lac-HEMA (DPav=6 and DS=10) is prepared in accordance with the procedure of step 1 and then sterilized in the dry state under UV light for 1 hr, in sterile PBS with 0.05% Irgacure® 2959.

A second solution of 13.3 w/v % of PVA-suc-lac-HEMA (DPav=4 and DS=10) is prepared in accordance with the procedure of step 2, then sterilized in the dry state under UV light for 1 hour, in sterile PBS with 0.05% Irgacure® 2959.

A gel precursor solution is formed by mixing equal volumes of the first solution and the second solution. If the hydrogels are to be made without added cells, then at this point the mixture can be dispensed into a 96-well plate, at 100 μL per well, and irradiated with UV light at a wavelength of 365 nm at 33 mW/cm² intensity for two minutes to initiate cross-linking, thereby causing the formation of hydrogels. The gels are washed twice, each wash being with 1 mL of phosphate buffered saline solution for ten minutes. The gels are then cultured in 1.5 mL of phosphate buffered saline.

Step 4. Inclusion of Cells

In some embodiments, it is desirable for the hydrogels to incorporate one or more therapeutic agents, and in some embodiments these therapeutic agents can be cells such as chondrocytes. In such embodiments, it is necessary to first prepare the chondrocytes for inclusion in the hydrogels.

a. Preparation of chondrocytes. In accordance with the following representative method, porcine joints are sprayed with 70% ethanol and allowed to soak for five minutes. Soft tissue is removed and the cartilage tissue exposed. The cartilage tissues are removed, minced into small particles and digested in 0.15% collagenase II solution (in DMEM/F12 containing 1% antibiotics) overnight, with about 0.3 g tissue per 10 mL solution. The digested cartilage suspension is then filtered through a 100 μm cell strainer. The filtrate is centrifuged at 2000 rpm for five minutes to obtain a cell pellet containing about 5 to 20 million cells.

b. Include cells in hydrogel. A pellet as prepared in step a. above was mixed with a desired quantity of the gel precursor solution prepared in step 3 above, and the cells were suspended uniformly. In this example, 0.6 mL of each of the first and second solutions were combined with a chondrocyte pellet to achieve a cell density of about 5 million cells per mL. The cell suspension was added to the 96 well plate at 100 μL per well. The suspensions were irradiated with UV light as described above for eight minutes to form gels. To each well 150 μL of cell culture medium is added to help remove the gels, then the gels are removed and transferred to a sterile 24-well plate, one gel per well. The gels are washed twice with 1 mL culture medium, for about 10 minutes per wash. Another 1.5 mL of medium is added to each gel in each well, and the gels are cultured for up to three weeks.

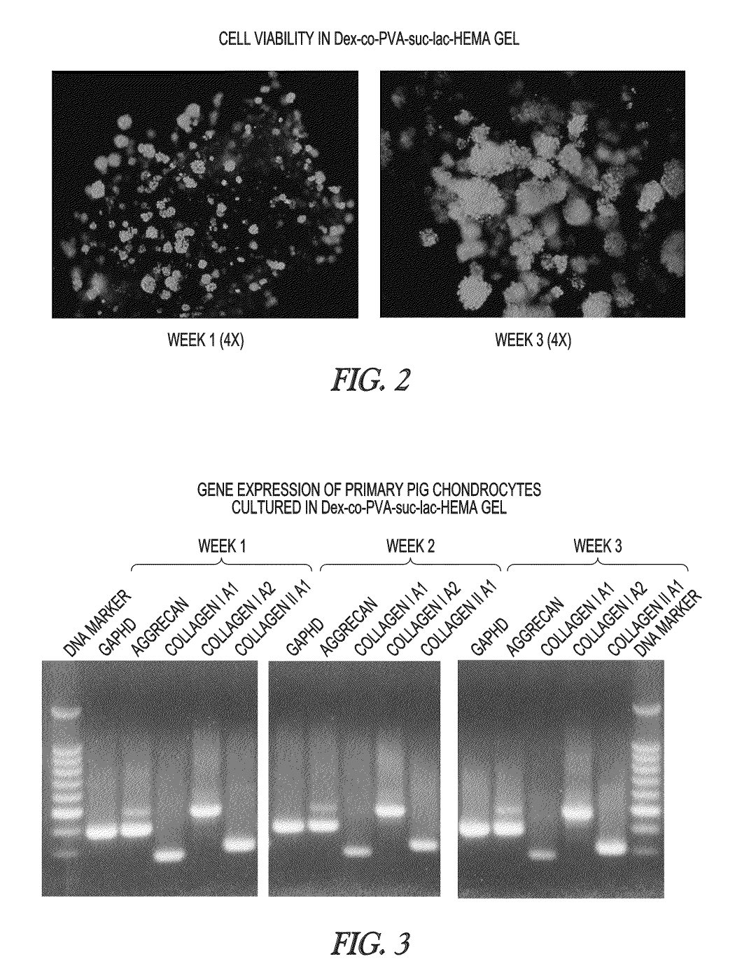

FIG. 2 is photographs of chondrocytes encapsulated in a hydrogel of the present invention after culturing for one week and three weeks. The photographs illustrate that the hydrogel of the invention contributes to the viability of the cells, and that they continue to grow after three weeks of incubation. FIG. 3 shows gene expression of chondrocytes encapsulated in a hydrogel of the present invention after culturing for one week, two weeks, and three weeks, as determined by reverse transcriptase-polymerase chain reaction (RT-PCR). The DNA markers are normal, indicating that the chondrocytes are viable and stable through three weeks of incubation. The data indicate that the chondrocytes marker genes are expressing genes of extracellular matrix components associated with viable chondrocytes expressed throughout the time range of the study.

### Example 2

A dextran-PVA hydrogel solution as described in Example 1 is used to encapsulate 4 million chondrocytes in 100 μL solution. Hydrogels are made from the solution by UV cross-linking at 80 mW/cm² for 1.5 minutes or at 33 mW/cm² for 2 minutes. The hydrogels so made are stored in a 24 well plate with 1.5 ml DMEM/F12 with 10% FBS (changed daily). Upon one week of culture, the sample is split for viability analysis using the Live/Dead® Viability/Cytotoxicity Kit of Invitrogen, biochemical analysis (stored at −20° C.) and histology/IHC (embedded in agarose, fixed with formulin). The results indicate that the cells are viable in each of the hydrogel samples.

Variations on the methods of obtaining chondrocytes will be understood by those skilled in the art. Joints from which chondrocyte are to be prepared should be stored on ice. In one method of obtaining chondrocytes, the joints are sprayed with 70% ethanol and allowed to sit for about 5 minutes. The joints are opened under sterile conditions, and menisci collected from the knee and/or cartilage. White zones are separated from the menisci and red zones are discarded. White zones are washed with phosphate buffered saline, then minced into small particles and digested with 0.2% collagenase I solution (in DMEM/F12 containing 1% antibiotics) overnight. Cartilage tissues are minced into small particles and digested in 0.15% collagenase II solution (in DMEM/F12 containing 1% antibiotics) overnight. In both cases, at least ten ml enzyme solution are used for each gram of tissue. After the digesting step, the menisci tissue suspension is filtered through a 70 μm cell strainer and the cartilage tissue suspension is filtered through a 100 μm cell strainer. The filtrate can be centrifuged at 2000 rpm for five minutes to obtain a cell pellet. The cells are resuspended in 1 mL medium and counted by Trypan blue exclusion for identifying the cell viability. There can be about 4,000,000 live cells and 60,000 dead cells. The cells can be spun down again to provide a pellet that can be used in hydrogel encapsulation. Other methods of isolating chondrocytes and preparing them for use in hydrogels will be familiar to shoes skilled in the art.

While the present invention has been illustrated by a description of various embodiments, and while these embodiments have been described in detail, it is not the intention of the inventors to restrict or in any way limit the scope of the appended claims to such detail. Additional advantages and modifications will readily appear to those skilled in the art. The various features of the invention may be used alone or in numerous combinations depending on the needs and preferences of the user.

What is claimed is:

1. A hydrogel-forming composition, comprising:
(a) a natural polymer derivative having a plurality of cross-linkable units depending therefrom, and
(b) a synthetic polymer derivative having a plurality of cross-linkable units depending therefrom, said natural and synthetic polymer derivatives having hydrolysable units disposed between the polymer backbone of said derivative and at least some of said cross-linkable units.

2. The hydrogel-forming composition of claim 1 wherein said cross-linkable units are selected from vinyl units, acrylate units, or mixtures thereof.

3. The hydrogel-forming composition of claim 1 wherein said natural polymer derivative is derived from a natural polymer selected from one or more of dextrans, polysaccharides, proteins such as gelatins and collagens, hyaluronic acid, and polyglycans.

4. The hydrogel-forming composition of claim 3 wherein said natural polysaccharide is dextran.

5. The hydrogel-forming composition of claim 1 wherein said synthetic polymer derivative is derived from a synthetic polymer selected from one or more of methyl cellulose, N-Isopropylacrylamide (NiPAAM); poly(vinyl alcohol); poly(NiPAAM)/poly(ethylene glycol); poly(ethylene oxidepropylene oxide-ethylene oxide) (PEO-PPO-PEO); and Poly(ethylene glycol-lactic acid-ethylene glycol) (PEG-PLLA-PEG).

6. The hydrogel-forming composition of claim 5 wherein said synthetic polymer is a polyvinylalcohol.

7. The hydrogel-forming composition of claim 1 wherein said hydrolysable units are selected from one or more of lactates, polyglycolic acid, polylactic acid, carbonate esters, carboxylic esters, urethanes, anhydrides, (hemi)acetals, amides, peptide bonds, and dimers and oligomers thereof.

8. The hydrogel-forming composition of claim 7 wherein said hydrolysable units comprise oligolactate.

9. The hydrogel-forming composition of claim 1 further comprising an aqueous medium.

10. The hydrogel-forming composition of claim 9 wherein said aqueous medium comprises a phosphate buffered saline solution.

11. The hydrogel-forming composition of claim 1 comprising one or more therapeutic agents.

12. The hydrogel-forming composition of claim 11 wherein one of the therapeutic agent comprises mammalian cells.

13. The hydrogel-forming composition of claim 1 which is subjected to cross-linking so as to form a cross-linked product.

14. A method of making a hydrogel composition, comprising:
(a) providing a natural polymer derivative having a plurality of cross-linkable units depending therefrom,
(b) providing a synthetic polymer derivative having a plurality of cross-linkable units depending therefrom, said natural and synthetic polymer derivatives having hydrolysable units disposed between the polymer backbone of said derivative and at least some of said cross-linkable units, and
(c) cross-linking said cross-linkable units depending from said natural polymer derivative with said cross-linkable units depending from said synthetic polymer derivative, so as to form a hydrogel composition.

15. The method of claim 14 further comprising providing an aqueous medium which is present during the cross-linking.

16. A method of administering a hydrogel composition to provide therapy to a site in a mammal in need thereof, comprising:
(a) providing a hydrogel-forming composition in accordance with claim 1, and
(b) administering said hydrogel-forming composition to said site.

17. The method of claim 16 further comprising initiating cross-linking of said hydrogel-forming composition after administration.

18. The method of claim 16 further comprising initiating cross-linking of said hydrogel-forming composition prior to administration to form a hydrogel composition.
